(19) United States
(12) Patent Application Publication
(10) Pub. No.: US 2026/0335275 A1
(43) Pub. Date: Oct. 29, 2026

(54) POLYPHENOLIC SUBSTANCE FORMULATION WITH ANTI-CANCEROGENIC EFFECT

(71) Applicant: ISTANBUL UNIVERSITESI REKTORLUGU, Istanbul (TR)

(72) Inventors: Oguz OZTURK, Fatih/Istanbul (TR); Hulya YILMAZ AYDOGAN, Fatih/Istanbul (TR); Mehmet Fatih SEYHAN, Fatih/Istanbul (TR); Allison Pinar ERONAT, Fatih/Istanbul (TR); Gulcin OZKARA, Fatih/Istanbul (TR); Ayse Begum CEVIZ, Fatih/Istanbul (TR)

(21) Appl. No.: 19/118,200

(22) PCT Filed: Sep. 26, 2023
(86) PCT No.: PCT/TR2023/051027

§ 371 Date: Apr. 3, 2025

(30) Foreign Application Priority Data
Oct. 3, 2022 (TR) 2022/015120

(51) Int. Cl. A61K 31/353; A61K 31/7048

(52) U.S. Cl. CPC A61K 31/353; A61K 31/7048

(57) ABSTRACT

The invention relates to a standardised polyphenolic substance formulation that has an anticarcinogenic effect, supports the effectiveness of standard treatment owing to its antioxidant, anti-inflammatory, hepatoprotective, cardioprotective, neuroprotective and systemic effects that are selectively cytotoxic against tumor cells, and has minimal side effects compared to the drugs that have anticarcinogenic, antioxidant and anti-inflammatory etc. properties in the state of the art when used at recommended doses. Mentioned formulation consists of combinations of comprising at least one of the natural substances including plant-derived curcumin, oleuropein, epigallocatechin gallate (EGCG), resveratrol, rosmarinic acid, carnosic acid, and caffeic acid phenethyl ester (CAPE), apigenin, galangin, chrysin, luteolin, quercetin and pinosembrin, which are found in various plants and propolis. The formulation that is the subject of the invention varies in active polyphenolic substance content depending on the type and the stage of the cancer it will be used in against. The formulation that is the subject of the invention is prepared in the form of suspension, syrup, sachet, tablet or capsule with excipients such as dextrose, maltose, maltodextrin or beta cyclodextrin in appropriate proportions in addition to the active ingredients.

### TECHNICAL FIELD OF THE INVENTION

The invention relates to a standardised polyphenolic substance formulation that has an anticarcinogenic effect, and supports the effectiveness of standard treatment owing to its antioxidant, anti-inflammatory, hepatoprotective, cardioprotective, neuroprotective and systemic effects that are selectively cytotoxic against tumor cells with minimal side effects compared to the drugs that have anticarcinogenic, antioxidant and anti-inflammatory etc. properties in the state of the art when used at recommended doses. Mentioned formulation consists of combinations comprising at least one of the natural substances including plant-derived curcumin, oleuropein, epigallocatechin gallate (EGCG), resveratrol, rosmarinic acid, carnosic acid, and caffeic acid phenethyl ester (CAPE), apigenin, galangin, chrysin, luteolin, quercetin and pinosembrin, which are found in both the plant and the propolis.

### STATE OF THE ART

Cancer is a multifactorial disease that occurs as a result of the accumulation of various mutations in many different cellular pathways in normal tissue and the uncontrolled increase of abnormal cells. Cancer cells are cells that have acquired various features that escape cellular control mechanisms, spread to neighbouring or distant tissues, and therefore cause uncontrolled growth. These cells develop mechanisms to ensure their continuity through many different cellular pathways. For example, breast cancer cells exhibit different metabolic phenotypes depending on their molecular subtype and metastatic site. Both intrinsic factors (MYC amplification, PIK3CA and TP53 mutations, etc.) and extrinsic factors (hypoxia, oxidative stress and acidosis, etc.) play a role in different metabolic reprogramming phenotypes in metastatic breast cancers. Cancer cells divide uncontrollably and irregularly, accumulate and form tumoral masses, and metastasise to surrounding tissues or to distant areas by breaking away from these cells and spreading into vascular access or body cavities. This situation appears as the most important handicap that reduces the success of cancer treatment.

Cancer treatment varies depending on the patient and cancer type. The most common treatment approaches are combinations of surgery, chemotherapy and radiotherapy. However, it may not be possible to remove all cancer cells with surgery. In addition, due to the possibility of cancer cells spreading to different tissues, surgical interventions alone are insufficient to eliminate all cancer cells. Radiotherapy, another treatment option that is often used together with surgery and chemotherapy, causes significant side effects on blood cells and surrounding tissues. In recent years, targeted immunotherapy and hormone therapy became available depending on the characteristics of the tumour. However, the lack of specific targeted treatment approaches for some types of cancer and the high rate of treatment resistance observed in some targeted treatments restrict the use and effectiveness of this type of treatment. For example, the effectiveness of monoclonal HER2 (human epidermal growth factor receptor 2) antibody treatments in HER2(+) (human epidermal growth factor receptor 2 (+)) breast cancer cases varies between 30-60% within the group. In the remaining cases, success is not achieved due to resistance mechanisms or side effects. In addition to all these, immunotherapy and hormone therapy, which are targeted treatments, can only be used when special markers are found in cancer cells, and this puts restrictions on the application of these treatment methods.

Another method used in cancer treatment is chemotherapy. More specifically, chemotherapy is a cancer treatment that uses one or more anticancer drugs as part of a standard treatment regimen. Chemotherapeutic agents used in chemotherapy are drugs that aim to kill cells by targeting various cellular mechanisms, preventing cells from dividing or causing DNA damage. However, these chemotherapeutic agents also have toxic effects on healthy cells. Due to these toxic effects, side effects occur at a high frequency in patients, patients may lose their lives due to chemotherapy alone, and different types of cancer may even develop in the future. On the other hand, due to certain genetic characteristics, some patients may be resistant to these agents and they have to undesirably interrupt the treatment due to nausea, fatigue and decreases in blood values. Due to these limitations and/or adverse effects, novel therapeutic approaches to support existing treatment approaches without any side effects are needed.

In the state of the art, there are many studies on therapeutic approaches that support cancer treatment approaches without side effects. The majority of these approaches consist of studies on breast cancer, one of the most common types of cancer in women. The fact that breast cancer, which is divided into subtypes, ranks second after lung cancer in terms of mortality rate encourages the majority of these studies. to focus on breast cancer. For this reason, studies on natural and synthetic substances that can have chemotherapeutic effects in the treatment of breast cancer, and also lung, prostate, colon and other organ cancers are increasing day by day. Many studies have taken their places the literature in this field, especially involving compounds and derivatives obtained from plants and used for cancer treatment. However, since mentioned compounds and their derivatives are often investigated using single or binary combinations, their mechanisms of action have not been sufficiently proven and only limited gene relationships have been studied.

Surgical intervention, chemotherapy, radiotherapy or combinations of these applications, which are included in standard cancer treatment approaches in the state of the art, may be insufficient to treat cancer with minimum damage, and success can be achieved in a very few anecdotal cases, especially in metastasized cancers. In summary, the treatment methods and combinations available in the state of the art are insufficient on their own, especially in the treatment of advanced stage cancers and in eliminating all cancer cells. Moreover, these mandatory treatments have toxic effects on other healthy cells and organs, side effects may occur and may even cause patient death. Immunotherapy and hormone treatments, which are targeted treatments, are effective only on cancer cells that carry special markers and to a limited extent. In addition to all these, in the state of the art, although efforts are being made to develop formulations that do not have side effects on cancer or breast cancer, these formulations are insufficient in terms of having no side effects, supporting standard treatment and increasing treatment effectiveness.

Due to reasons such as the limitations and inadequacies of cancer treatment applications in the state of the art, and the facts that applications such as surgical intervention, chemotherapy, radiotherapy, immunotherapy and hormone treatments are not sufficient alone in the treatment of cancer, and that these applications cause additional damage to the body and in some cases may even cause patient deaths, and that they do not have the effectiveness to minimise the toxic effect in cancer treatment, it has become necessary to develop a formulation that supports the treatment applications in the state of the art and even increases the effectiveness of these applications and reduces their side effects, as well as being anticarcinogenic and minimizing side effects.

### BRIEF DESCRIPTION AND AIMS OF THE INVENTION

The presented invention is a standardised polyphenolic substance formulation with an anticarcinogenic effect that supports the effectiveness of standard treatment by means of its antioxidant, anti-inflammatory, hepatoprotective, cardioprotective, neuroprotective and systemic effects that are selectively cytotoxic against tumor cells, and has minimal side effects compared to the drugs that have anticarcinogenic, antioxidant and anti-inflammatory etc. properties in the state of the art when used at recommended doses, is explained. Mentioned formulation consists of combinations of comprising at least one of the natural substances including plant-derived curcumin, oleuropein, epigallocatechin gallate (EGCG), resveratrol, rosmarinic acid, carnosic acid, and caffeic acid phenethyl ester (CAPE), apigenin, galangin, chrysin, luteolin, quercetin and pinosembrin, which are found in both various plants and propolis.

One of the aims of the invention is to provide a formulation that has an anticarcinogenic effect, supports standard treatment, increases the effectiveness of standard treatment, and has minimal side effects compared to drugs that have anticarcinogenic, antioxidant and anti-inflammatory properties, etc., in the state of the art when used at recommended doses. In the invention, a formulation with mentioned properties is provided by ensuring that the formulation comprises at least one or more of apigenin, galangin, caffeic acid phenethyl ester (CAPE), chrysin, curcumin, epigallo catechingallate (EGCG), luteolin, oleuropein, quercetin, resveratrol, rosmarinic acid, carnosic acid and pinosembrin in specific ratios. Given ingredients are obtained entirely from natural sources and consist of nutraceutical and/or pharmaceutical grade raw materials. These polyphenolic substances, which are already included in daily human nutrition in certain amounts, do not have obvious or significant side effects at certain dosage ranges. The formulation of the invention is prepared with a multi-substance approach at relatively low doses, and by affecting different cellular pathways simultaneously, it prevents the tendency of cancer cells turning to alternative pathways, which is frequently encountered, and thus has a detrimental effect on the survival of cancer cells. In other words, by means of the formulation of the invention, the facilitation of the assimilation of carbon into macromolecules as a result of oncogenic signaling pathways directing nutrient uptake is prevented, and therefore the growth and proliferation of cancer cells is prevented.

Another aim of the invention is to provide a standardised polyphenolic substance formulation with a high level of anticarcinogenic effect. The provision of the formulation with a high level of anticarcinogenic effect is provided by ensuring that the formulation comprises at least one or more of apigenin, galangin, caffeic acid phenethyl ester (CAPE), chrysin, curcumin, epigallo catechingallate (EGCG), luteolin, oleuropein, quercetin, resveratrol, rosmarinic acid, carnosic acid and pinosembrin in specific ratios. High anticarcinogenic activity is achieved by using the formulation that is the subject of the invention in percentages determined according to the stage of the cancer and the clinical condition of the patient.

Another aim of the invention is to provide a formulation that highly supports/increases the effectiveness of standard cancer treatment. A formulation that is highly supportive of standard cancer treatments is provided by ensuring that the formulation that is the subject of the invention comprises at least one or more of apigenin, galangin, caffeic acid phenethyl ester (CAPE), chrysin, curcumin, epigallo catechin gallate (EGCG), luteolin, oleuropein, quercetin, resveratrol, rosmarinic acid, carnosic acid and pinosembrin in specific ratios. While the formulation of the invention shows an anticarcinogenic effect on breast cancer types, it does not have a significant cytotoxic effect on breast fibro-epithelial cells used as control and therefore has the power to provide additional benefit to standard cancer treatment.

Another aim of the invention is to provide a formulation that reduces the excessive oxidation activity that may occur with standard cancer treatment, which can be extremely toxic to healthy tissues, and thus reduces the side effects of standard treatment. A formulation that reduces the side effects of standard treatment without reducing its anticancer effectiveness is provided by ensuring that the formulation that is the subject of the invention comprises at least one or more of apigenin, galangin, caffeic acid phenethyl ester (CAPE), chrysin, curcumin, epigallo catechin gallate (EGCG), luteolin, oleuropein, quercetin, resveratrol, rosmarinic acid, carnosic acid and pinosembrin in specific ratios.

### DESCRIPTION OF DRAWINGS

FIG. 1. Graph showing the cytotoxic effect of a apigenin-chrysin-galangin formulation on MCF-7 cell line

FIG. 2. Graph showing the cytotoxic effect of a apigenin-chrysin-galangin formulation on MCF-10A cell line

FIG. 3. Graph showing the cytotoxic effect of a catechin formulation on MCF-7 cell

FIG. 4. Graph showing the cytotoxic effect of a catechin formulation on MCF-10A cell

FIG. 5. Graph showing the cytotoxic effect of a flavonoid formulation on MCF-7 breast cancer cell line

FIG. 6. Graph showing the cytotoxic effect of the a flavonoid formulation on the control MCF-10A cell line

FIG. 7. Cell cycle analysis of control group MCF-7 cells

FIG. 8. 24^{th} hour cell cycle analysis of MCF-7 cells treated with a 10% flavonoid formulation

FIG. 9. 48^{th} hour cell cycle analysis of MCF-7 cells treated with a 10% flavonoid formulation

FIG. 10. Fold change histogram graph of Aurora kinase A (AURKA) and Aurora kinase B (AURKB) protein expressions in MCF-7 cells treated with flavonoid formulation compared to control

FIG. 11. Graph showing the dose-time dependent cytotoxic effects of a flavonoid formulation on HER2(+)/SKBR-3 breast cancer cell line

FIG. 12. Graph showing the dose-time dependent cytotoxic effects of the polyphenolic substance formulation of the invention on the HER2(+)/SKBR-3 breast cancer cell line

FIG. 13. Graph showing the dose-time dependent cytotoxic effects of the polyphenolic substance formulation of the invention on the triple negative/MDA-MB-231 cell line

FIG. 14. Graph showing the dose-time dependent cytotoxic effects of the polyphenolic substance formulation of the invention on the fibrocystic MCF-10A breast epithelial cell line

FIG. 15. Graph showing the dose-time dependent cytotoxic effects of the polyphenolic substance formulation of the invention on the ER(+)/PR(+) MCF-7 breast cancer cell line

FIG. 16. Inverted light microscopy images showing the effects of the polyphenolic substance formulation of the invention on cell migration on the MDA-MB-231 cell line.

FIG. 17. Comparison of the effect of the polyphenolic substance formulation of the invention on human peripheral blood mononuclear cells (PBMC) and its effectiveness against the division stimulus (PHA).

### DETAILED DESCRIPTION OF THE INVENTION

The invention relates to a standardised polyphenolic substance formulation that has an anticarcinogenic effect, supports the effectiveness of standard treatment by means of especially its antioxidant, anti-inflammatory, hepatoprotective, cardioprotective, neuroprotective and systemic effects that are selectively cytotoxic against tumor cells, and has minimal side effects compared to the drugs that have anticarcinogenic, antioxidant and anti-inflammatory etc. properties in the state of the art when used at recommended doses. Mentioned formulation consists of combinations of comprising at least one of the natural substances including plant-derived curcumin, oleuropein, epigallocatechin gallate (EGCG), resveratrol, rosmarinic acid, carnosic acid, and caffeic acid phenethyl ester (CAPE), apigenin, galangin, chrysin, luteolin, quercetin and pinosembrin, which are found in both the plants and propolis. The formulation that is the subject of the invention varies in active polyphenolic substance content depending on the type of cancer it will be used against and the stage of the cancer. The formulation that is the subject of the invention is prepared in the form of suspension, syrup, sachet, tablet or capsule with excipients such as dextrose, maltose, maltodextrin or beta cyclodextrin in appropriate proportions in addition to the active ingredients.

The formulation that is the subject of the invention varies in active polyphenolic substance content depending on the type and the stage of the cancer it will be used against. The formulation that is the subject of the invention is prepared in the form of suspension, syrup, sachet, tablet or capsule with excipients such as dextrose, maltose, maltodextrin or beta cyclodextrin in appropriate proportions in addition to the active ingredients.

The polyphenolic substance formulation that is the subject of the invention, which has an anticarcinogenic effect, kills cancer cells and provides an increase in immune system cells by synergistically affecting many genes involved in cell division, programmed cell death and many other pathways.

The polyphenolic substance formulation of the invention can be used against various cancers by changing the content ratios. The daily treatment dose should be 10-200 mg/kg/day total phenolic substance content, depending on the type and the stage of the disease. The ratios of polyphenols in the mixture as minimum and maximum percentage (by mass) are given in Table 1.

TABLE 1
Ratios of active ingredients contained in the polyphenolic
substance formulation that is the subject of the invention

| | Percentage in the |
|---|---|
| | mixture (by mass) |
| Apigenin | 1-10% |
| Galangin | 1-10% |
| Caffeic acid phenethyl ester (CAPE) | 1-10% |
| Krisin | 1-10% |
| Curcumin | 1-10% |
| Epigallo catechin gallate (EGCG) | 1-10% |
| Luteolin | 1-10% |
| Oleuropein | 1-10% |
| Quercetin | 1-10% |
| Resveratrol | 1-10% |
| Carnosic acid | 1-10% |
| Rosmarinic acid | 1-10% |
| Pinosembrin | 1-10% |
| Total | 100% |

The formulation that is the subject of the invention varies in active polyphenolic substance content depending on the type and the stage of the cancer it will be used against. The formulation that is the subject of the invention is prepared in the form of suspension, syrup, sachet, tablet or capsule with excipients such as dextrose, maltose, maltodextrin or beta cyclodextrin in appropriate proportions in addition to the active ingredients.

In one embodiment of the invention, the formulation according to the invention comprises at least three of the apigenin, galangin, caffeic acid phenethyl ester (CAPE), chrysin, curcumin, epigallo catechin gallate (EGCG), luteolin, oleuropein, quercetin, resveratrol, carnosic acid, rosmarinic acid and pinosembrin.

In one embodiment of the invention, the formulation according to the invention comprises at least four of the apigenin, galangin, caffeic acid phenethyl ester (CAPE), chrysin, curcumin, epigallo catechin gallate (EGCG), luteolin, oleuropein, quercetin, resveratrol, carnosic acid, rosmarinic acid and pinosembrin. Said formulation comprises apigenin, galangin, chrysin and europein.

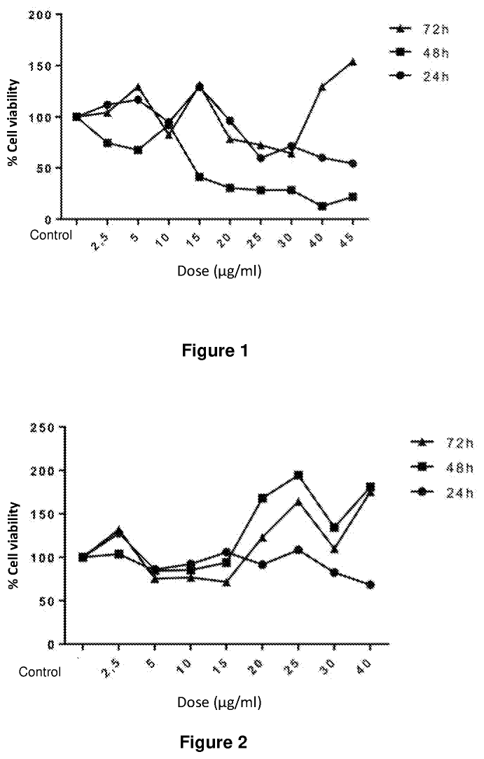

FIGS. 1-17 shows the activities of the polyphenolic mixtures of the invention on cell lines representing various types of breast cancer as an example disease group.

FIG. 1 is the graph showing the cytotoxic effect of the apigenin-chrysin-galangin formulation on the MCF-7 cell line, and FIG. 2 is the graph showing the cytotoxic effect of the apigenin-chrysin-galangin formulation on the MCF-10A cell line. The combined effects of some flavonoids were examined on the MCF-7 cell line, which models invasive ductal ER/PR (+) breast cancer, the most common type of breast cancer, and on the MCF-10A cell line, which models fibrocystic breast epithelium as a control. A significant cytotoxic effect of the apigenin-chrysin-galangin formulation on MCF-7 cells was detected, especially at the 48^{th} hour, starting from low doses (15 μg/mL) and continuing up to the highest doses, and this effect was confirmed to be apoptotic by the Annexin FITC assay. No cytotoxic effect of this formulation was detected on control MCF-10A cells, reducing cell viability.

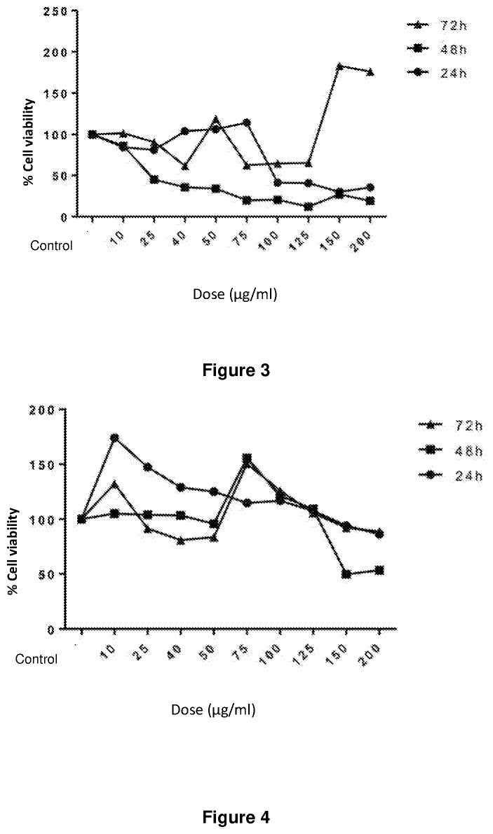

FIG. 3 shows the graph showing the cytotoxic effect of the catechin formulation on the MCF-7 cell, and FIG. 4 shows the cytotoxic effect of the catechin formulation on the MCF-10A cell. Similar effects of the prepared flavanol (catechin) formulation to the apigenin-chrysin-galangin formulation were detected in MCF-7 breast cancer cells and the cytotoxic effect of said catechin formulation continued to increase from very low doses (25 μg/mL) to the highest dose (in 50% of the cells), especially at the 48^{th} hour, and it was confirmed by Annexin VI experiments that the effect was of apoptotic origin. In cytotoxicity experiments, statistically significant results were obtained starting from the 100 μg/mL dose at the 24^{th} hour and the 50 μg/mL dose at the 72^{nd} hour. Similar to the apigenin-chrysin-galangin formulation, no cytotoxic effect of the catechin formulation was observed on MCF-10A cells.

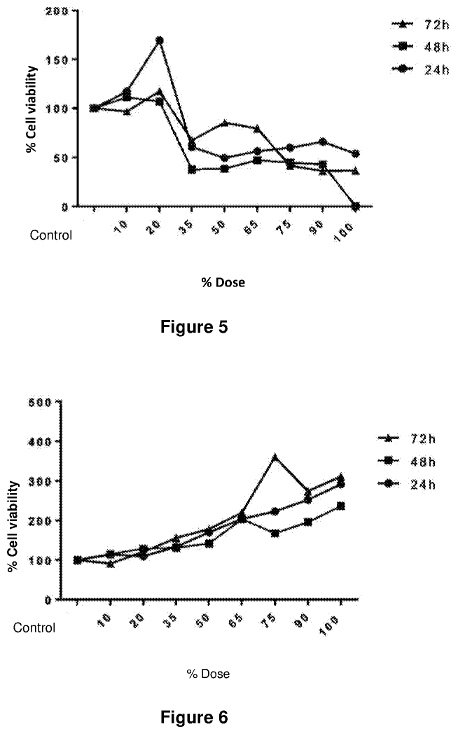

FIG. 5 is the graph showing the cytotoxic effect of the flavonoid formulation on the MCF-7 breast cancer cell line, and FIG. 6 is the graph showing the cytotoxic effect of the flavonoid formulation on the control MCF-10A cell line. Significant effects of said flavonoid formulation on cell viability/toxicity were observed in the MCF-7 breast cancer cell line. The flavonoid formulation caused a dramatic, albeit statistically significant, decrease in cell viability in MCF-7 cells at a dose of 35% in the early and later stages (24^{th}, 48^{th} and 72^{nd} hours). Additionally, no cytotoxic effect of the flavonoid formulation was observed at any time and dose on fibrocystic breast epithelial MCF-10A cells used as a control, as shown in FIG. 6.

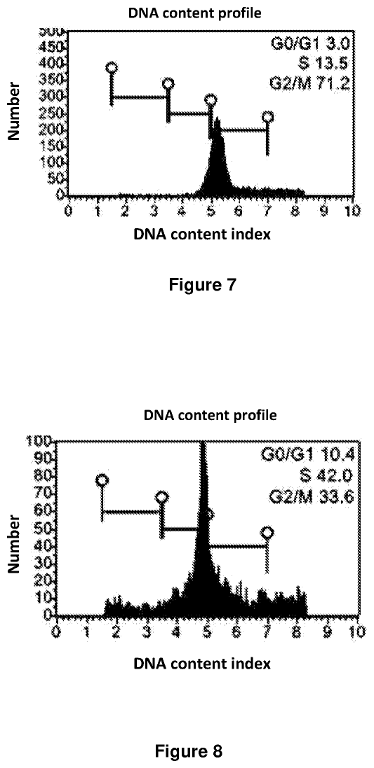

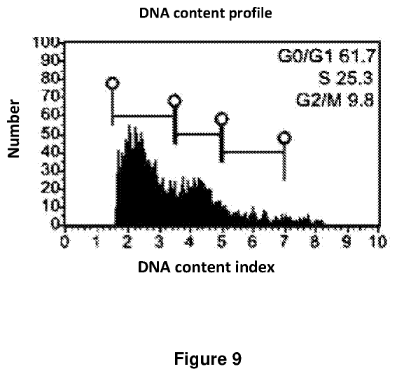

FIG. 7 shows the cell cycle analysis of control group MCF-7 cells, FIG. 8 shows the 24^{th} hour cell cycle analysis of MCF-7 cells to which 10% flavonoid formulation was applied, and FIG. 9 shows the 48^{th} hour cell cycle analysis of MCF-7 cells to which 10% flavonoid formulation was applied. In the cell cycle analysis performed, it was observed that 70% of the cells in the MCF-7 cell line, to which flavonoid formulation was not applied, were in the G2/M phase, in other words, they were in the actively dividing phase, and by applying a 10% dose of flavonoid formulation to MCF-7 cells, it was observed that MCF-7 cells were arrested in the S phase in the first 24 hours, and a significant majority of the surviving cells were arrested in the G0/G1 phase at the 48^{th} hour, in other words, the polyphenolic substance formulation of the invention inhibited the proliferation of cancer cells.

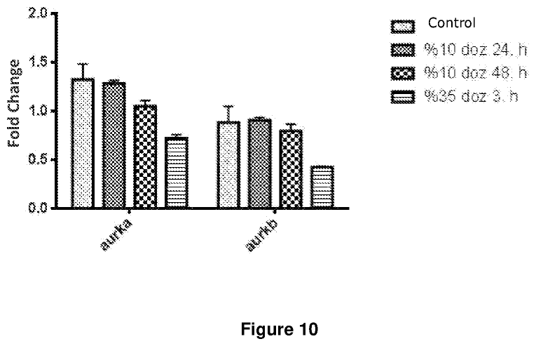

FIG. 10 shows the fold change histogram graph of Aurora kinase A (AURKA) and B (AURKB) protein expressions in MCF-7 cells treated with flavonoid formulation compared to the control. A significant decrease in the expression of both Aurora kinase A and Aurora kinase B was detected in MCF-7 cells to which the flavonoid formulation was applied. Aurora kinases are mitotic kinases that are highly expressed in most cancers and are responsible for regulating many events including centrosome maturation, entry into mitosis, centrosome separation, bipolar spindle assembly, alignment of chromosomes in metaphase, and cytokinesis. Therefore, the expressions of these two enzymes at the protein level were analysed using the “protein immunoblotting method” and it was confirmed that the expression of both proteins was significantly reduced. In other words, the formulation of the invention reduces the expression of Aurora kinase A and B proteins and is shown to have a suppressive effect on cell division.

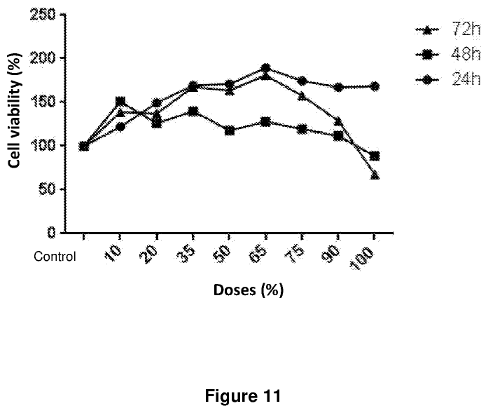

The dose-time dependent cytotoxic effects of progressive flavonoid formulation studies on the HER2(+) (human epidermal growth factor receptor 2 (+)) breast cancer cell line are shown in FIG. 11. As can be clearly seen from the graph in FIG. 11, mentioned flavonoid formulation was not effective enough on SKBR-3 cells, which model the more aggressive HER2(+) breast cancer, the frequency of which is increasing in societies, so the effectiveness was achieved by enriching the formulation.

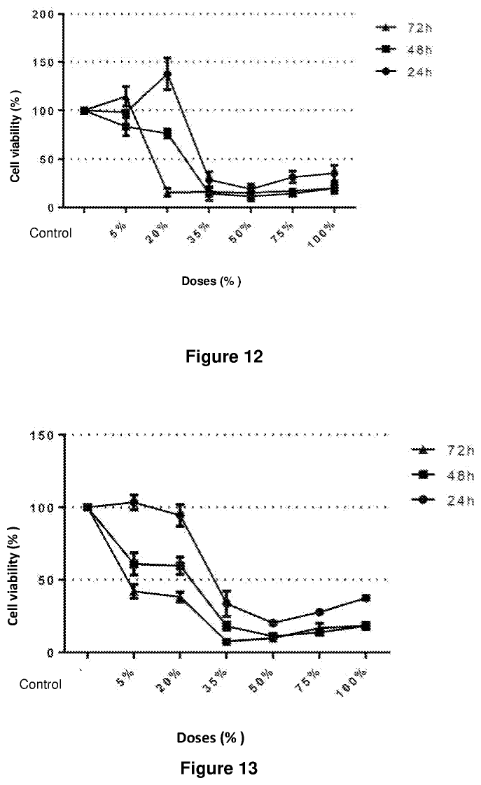

As a result of all these reasons explained and supported by figures, a formulation that may be effective in MDA-MB-231 cell line modelling triple negative (ER/PR (−) and HER2(−)) types of breast cancer that are more resistant to chemotherapy and have higher recurrence rates and SKBR3 cell line modeling the HER2(+) (ER/PR(−) and HER2(+)) type and in adenocarcinomas of other tissues is provided with the polyphenolic substance formulation that is the subject of the invention. In light of the findings obtained from cytotoxicity analyses, it has been determined that the formulation that is the subject of the invention is also effective in aggressive types of breast cancer. Mentioned activity is clearly understood from the graph in FIG. 12 showing the dose-time dependent cytotoxic effects of the polyphenolic substance formulation of the invention on the HER2(+)/SKBR-3 breast cancer cell line and from the graph in FIG. 13 showing the dose-time dependent cytotoxic effects of the polyphenolic substance formulation of the invention on the triple negative/MDA-MB-231 cell line.

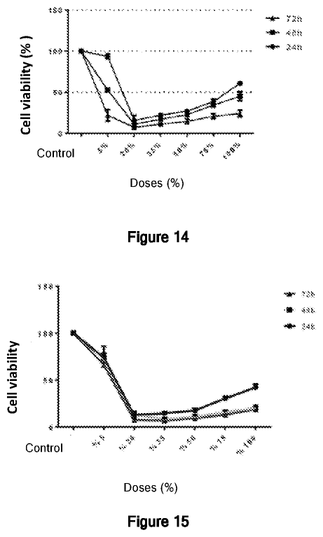

FIG. 14 gives the graph showing the dose-time dependent cytotoxic effects of the polyphenolic substance formulation of the invention on the fibrocystic MCF-10A breast epithelial cell line, and FIG. 15 gives the graph showing the dose-time dependent cytotoxic effects of the polyphenolic substance formulation of the invention on the ER(+)/PR(+) MCF-7 breast cancer cell line. Cytotoxic effects of the polyphenolic substance formulation that is the subject of the invention were determined on the ER(+)/PR(+) MCF-7 breast cancer cell line, which was confirmed to be apoptotic-induced by Annexin V, starting from low doses and early stages. The cytotoxic effect of the polyphenolic substance formulation of the invention was also observed on MCF-10A control cells. As we described previously, MCF-10A cells are non-tumourogenic fibrocystic breast epithelial cells. All these results show that the polyphenolic substance formulation of the invention is effective not only in hormone-positive and hormone-negative breast cancers but also in fibrocystic changes of the breast tissue.

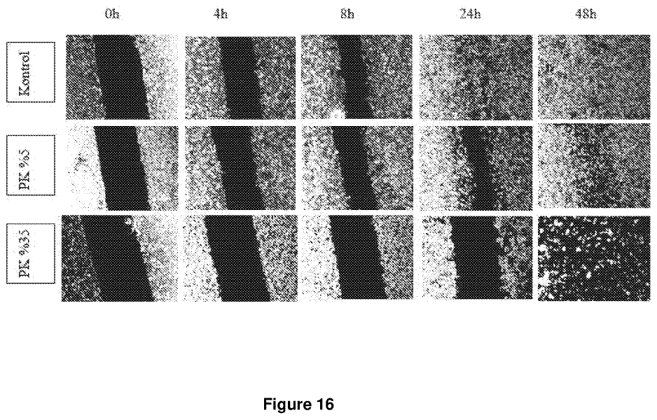

As a result, the evaluation of the effects of the polyphenolic substance formulation that is the subject of the invention observed at different stages of different cell lines by cell cycle analysis shows that the mentioned formulation is effective at different points of the cell cycle, and PCR array analyses show that the formulation that is the subject of the invention is effective on many pathways related to cell cycle arrest, especially in the G0/G1 phase, and induction of apoptosis. Given result was obtained by counting apoptotic, necrotic and living cells in the “cell cycle analysis” and in the apoptosis analysis performed using Annexin V-propidium iodine on the flow cytometry device. In addition, when the effect of the polyphenolic substance formulation of the invention on cell migration was examined in the scratch test on metastatic and hormone negative MDA-MB-231 cells, it was seen that the formulation of the invention had a dose-dependent reducing effect on cell migration. Since cell migration experimentally models metastasis, decreased migration indicates that the metastatic property of cells also decreases. It has been determined and proven that a 35% dose of the formulation that is the subject of the invention stops the migration of cells until the 48^{th} hour and has a cytotoxic effect on all cells at the 48^{th} hour. FIG. 16 shows images taken under an inverted light microscope showing the effects of the polyphenolic substance formulation of the invention on cell migration on the MDA-MB-231 cell line.

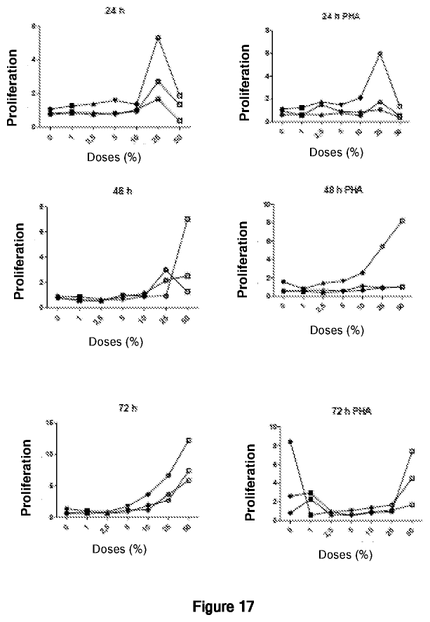

It has been determined that the polyphenolic substance formulation of the invention increases the durability and survival rate of human peripheral blood mononuclear cells (PBMC) and suppresses the increase in immune system cells due to external stimuli (phytohemagglutinin (PHA)). This shows that the formulation of the invention also strengthens the immune system in a controlled manner. These effects are clearly seen in the graph in FIG. 17 comparing the effect of the polyphenolic substance formulation of the invention on human peripheral blood mononuclear cells (PBMC) and its effectiveness against the division stimulus (PHA).

In short, the fact that mentioned ingredients are brought together in specific proportions in the invention gives the formulation of the invention a synergistic effect that is effective on cancer. The formulation of the invention is effective on MDA-MB-231 and SKBR3 cell lines, which model breast cancer types that are more resistant to chemotherapy and have higher recurrence rates. In addition, said polyphenolic substance formulation is also effective in aggressive types of breast cancer. In addition to all these, mentioned formulation has a cytotoxic effect on the MCF-7 breast cancer cell line from low doses and early stages, and also has a cytotoxic effect on fibrocystic MCF-10A cells used as a control. The polyphenolic substance formulation of the invention increases the durability and survival rate of human peripheral blood mononuclear cells (PBMC) and suppresses the increase in immune system cells due to external stimuli (phytohemagglutinin (PHA)). Therefore, the formulation of the invention also strengthens the immune system in a controlled manner. The formulation of the invention is effective on various adenocarcinomas (respiratory system, digestive system and urogenital system cancer, etc.), especially breast cancers, which go through similar carcinogenesis pathways and show similar gene expressions.

1. An anticarcinogenic polyphenolic substance formulation, comprising at least one of apigenin, galangin, caffeic acid phenethyl ester (CAPE), chrysin, curcumin, epigallo catechin gallate (EGCG), luteolin, oleuropein, quercetin, resveratrol, carnosic acid, rosmarinic acid and pinosembrin.

2. The formulation according to claim 1, comprising at least one of 1-10% apigenin, 1-10% galangin, 1-10% caffeic acid phenethyl ester (CAPE), 1-10% chrysin, 1-10% curcumin, 1-10% epigallo catechin gallate (EGCG), 1-10% luteolin, 1-10% oleuropein, 1-10% quercetin, 1-10% resveratrol, 1-10% carnosic acid, 1-10% rosmarinic acid and 1-10% pinosembrin by mass.

3. The formulation according to claim 1, comprising at least three of apigenin, galangin, caffeic acid phenethyl ester (CAPE), chrysin, curcumin, epigallo catechin gallate (EGCG), luteolin, oleuropein, quercetin, resveratrol, carnosic acid, rosmarinic acid and pinosembrin.

4. The formulation according to claim 1, comprising at least four of apigenin, galangin, caffeic acid phenethyl ester (CAPE), chrysin, curcumin, epigallo catechin gallate (EGCG), luteolin, oleuropein, quercetin, resveratrol, carnosic acid, rosmarinic acid and pinosembrin.

5. The formulation according to claim 4, comprising apigenin, galangin, chrysin and ouropein.

6. The formulation according to claim 1, wherein the formulation is in the form of suspension, syrup, sachet, tablet or capsule.

7. The formulation according to claim 6, comprising 1-10% apigenin, 1-10% galangin, 1-10% caffeic acid phenethyl ester (CAPE), 1-10% chrysin, 1-10% curcumin, 1-10% epigallo catechin gallate (EGCG), 1-10% luteolin, 1-10% oleuropein, 1-10% quercetin, 1-10% resveratrol, 1-10% carnosic acid, 1-10% rosmarinic acid and/or 1-10% pinosembrin by mass.

8. The formulation according to claim 1, wherein the daily treatment dose is 10-200 mg/kg/day.
